(19) United States
(12) Patent Application Publication
(10) Pub. No.: US 2009/0312841 A1
(43) Pub. Date: Dec. 17, 2009

(54) BONE VOID FILLER

(75) Inventor: Michael J. LaLonde, Warsaw, IN (US)

Correspondence Address: ZIMMER TECHNOLOGY - BAKER & DANIELS, 111 EAST WAYNE STREET, SUITE 800, FORT WAYNE, IN, 46802, US

(73) Assignee: ZIMMER, INC., Warsaw, IN (US)

(21) Appl. No.: 12/138,904

(22) Filed: Jun. 13, 2008

(51) Int. Cl. A61F 2/28

(52) U.S. Cl. 623/23.48

(57) ABSTRACT

A plug for filling a bone void, such as a void left behind after implant removal, and a method for using the same. The plug is strong and durable enough to increase the strength of the void and the bone surrounding the void upon insertion. The plug's porous, open-cell construction may increase the strength of the void and the bone surrounding the void by permitting bone growth into the plug. The plug may be provided with a channel configured to receive a bone growth promoting material, a medication, or another material.

### BACKGROUND

1. Field of the Invention

The present invention relates to filling voids in bone. More particularly, the present invention relates to porous plugs for filling voids in bone, and to a method for using the same.

2. Description of the Related Art

Over time an implant may no longer be needed in a patient's body. For example, a plate and screws implanted in the patient's body to support a fractured bone may no longer be needed when the fracture heals. A surgeon may decide to leave the implant in place or to remove the implant. There are risks associated with both options. If the surgeon chooses to leave the implant in place, the patient may suffer symptoms due to, for example, an infection. Even asymptomatic implants have been known to cause the bone to refracture, especially among active patients. Either occurrence may eventually require that the surgeon remove the implant. Removing the implant, on the other hand, presents its own risks. For one, the patient will be subjected to the general risks of surgery. Also, removing the implant causes some trauma to the bone, even if performed successfully. Finally, removing the implant leaves behind voids in the bone once filled by anchors, such as screws. Together with the general trauma caused by screw removal, the voids in the bone act as stress risers, significantly weakening the bone and possibly causing the bone to refracture.

Researchers have proposed many methods for filling voids in the bone after implant removal. For example, researchers have proposed over-drilling the voids to cause bleeding and stimulate new bone growth. Others have proposed filling the voids with grafted bone and/or solid plugs. Still others have proposed injecting cement into the void.

### SUMMARY

The present invention provides a plug for filling a bone void, such as a void left behind after implant removal. The plug is strong and durable enough to increase the strength of the void and the bone surrounding the void upon insertion. The plug's porous, open-cell construction may increase the strength of the void and the bone surrounding the void by permitting bone growth into the plug. The plug may be provided with a channel configured to receive a bone growth promoting material, medication, or another material. The present invention also provides a method for using the plug to fill the void.

According to an embodiment of the present invention, a plug is provided for filling a bone void. The plug has a distal end, a proximal end, and a shaft extending between the proximal end and the distal end of the plug. The plug further includes a thread that extends from the shaft and wraps helically around the shaft. The plug includes a porous, open-cell material.

According to another embodiment of the present invention, a plug is provided for filling a bone void. The plug has a distal end defining a terminal end of the plug and a proximal end defining another terminal end of the plug. The plug also includes a shaft sized to be received within the void. The shaft extends between the distal end and the proximal end, whereby the plug lacks a head that extends radially outwardly beyond the shaft. The plug further includes a thread that extends from the shaft and wraps helically around the shaft. The plug includes a porous, open-cell material.

According to yet another embodiment of the present invention, a method is provided for filling a void in a bone. The method involves providing access to the void and filling the void with a plug constructed of a porous, open-cell material and sized to be received within the void. The plug includes a proximal end, a distal end, a shaft extending between the proximal end and the distal end, and a helical thread.

### BRIEF DESCRIPTION OF THE DRAWINGS

The above-mentioned and other features and advantages of this invention, and the manner of attaining them, will become more apparent and the invention itself will be better understood by reference to the following description of embodiments of the invention taken in conjunction with the accompanying drawings, wherein:

FIG. 1 is an elevational view of a tibia with a partial cross-section to illustrate a void;

FIG. 2 is a perspective view of an embodiment of a plug;

FIG. 3 is a cross-sectional view of the plug of FIG. 2 taken along line 3-3 of FIG. 2;

FIG. 4 is a perspective view of another embodiment of a plug having a channel configured to receive an insert;

FIG. 5 is a cross-sectional view of the plug of FIG. 4; and

FIG. 6 is a partial view of the tibia of FIG. 1 with a plug in the void.

Corresponding reference characters indicate corresponding parts throughout the several views. The exemplifications set out herein illustrate exemplary embodiments of the invention and such exemplifications are not to be construed as limiting the scope of the invention any manner.

### DETAILED DESCRIPTION

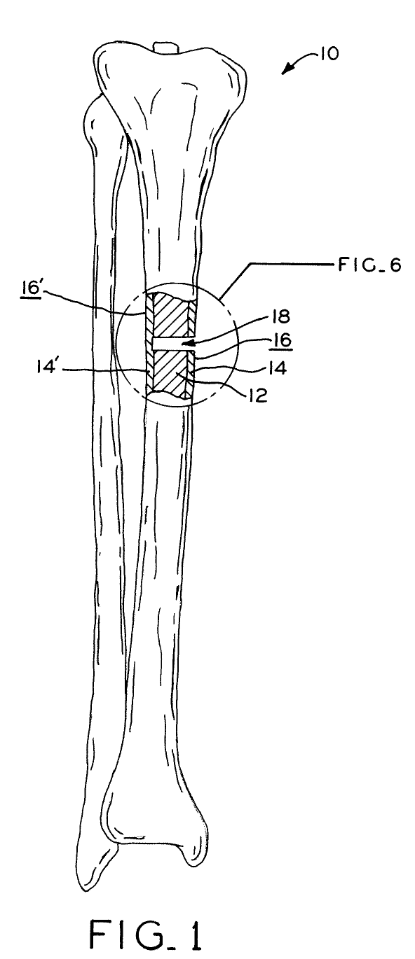

Referring to FIG. 1, a bone is illustrated in the form of tibia 10. Although the bone is illustrated and described herein as tibia 10, the bone may include another bone of the body in accordance with the teachings herein. For example, the bone may include a femur, a pelvis, a humerus, an ulna, a radius, a clavicle, or another bone of the body. Tibia 10 includes cancellous layer 12 surrounded by cortical layer 14. The bone of cancellous layer 12 is soft and spongy compared to the bone of cortical layer 14. Tibia 10 further includes cortical surface 16, an exterior surface of cortical layer 14.

Referring still to FIG. 1, tibia 10 includes void 18. Void 18 may be caused by, for example, removal of an implant. Specifically, void 18 may be caused by removal of an anchor of the implant, such as a bone screw. As illustrated, the scale of void 18, especially the diameter of void 18, relative to the scale of tibia 10 may be exaggerated.

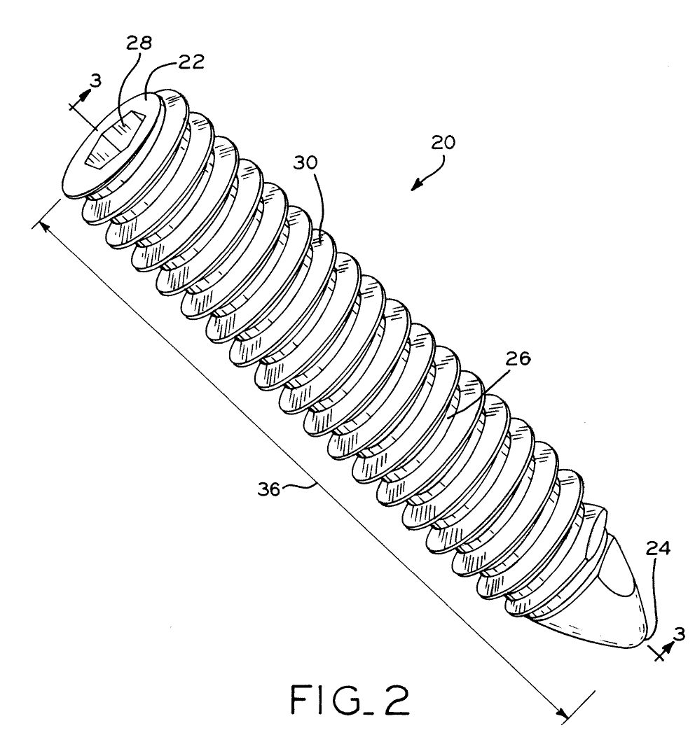

Plug 20, illustrated in FIGS. 2-5, is provided to fill void 18 in tibia 10 (FIG. 1). Plug 20 includes proximal end 22 and distal end 24. As used herein, “proximal” and “distal” are determined relative to a surgeon or another user, such that distal end 24 of plug 20 is farther from the user than proximal end 22 of plug 20. Plug 20 further includes shaft 26 that extends between proximal end 22 and distal end 24. Proximal end 22 of plug 20 includes bore 28 that cooperates with a tool (not shown) for turning plug 20. Bore 28 may be hexagonal to receive a hex wrench, also known as an Allen wrench. Bore 28 may also be D-shaped, slotted, star-shaped, or another known shape, and configured to cooperate with a similarly shaped tool. Distal end 24 of plug 20 tapers to a point, permitting plug 20 to be tapped into bone surrounding void 18.

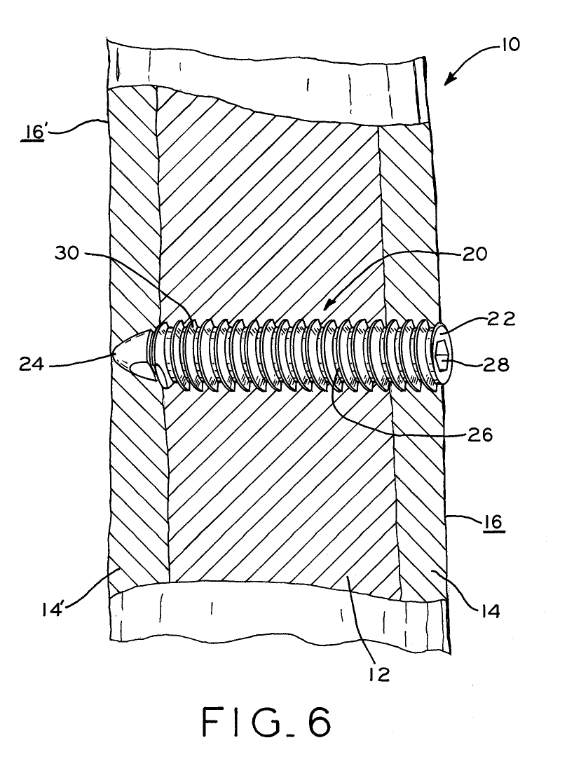

Unlike a typical screw, proximal end 22 of plug 20 lacks a head extending outwardly beyond shaft 26. After widening near distal end 24, shaft 26 of plug 20 extends to proximal end 22 with an essentially uniform diameter. The absence of a head permits plug 20 to be driven substantially in line with or beneath cortical surface 16 of tibia 10 (FIG. 6). If plug 20 were provided with a head, the head would rest outside of void 18 against cortical surface 16 of tibia 10. In this position, the head of plug 20 could wear against surfaces adjacent to tibia 10, including soft tissue.

Referring still to FIGS. 2-5, shaft 26 of plug 20 includes thread 30 that extends from shaft 26 and wraps helically around shaft 26. Thread 30 may extend length 36 of shaft 26, from proximal end 22 to distal end 24, or a portion thereof. Thread 30 is interrupted near tapered distal end 24, permitting plug 20 be to tapped into bone surrounding void 18, including bone of cortical layer 14.

Referring to FIGS. 1 and 6, a method is provided for filling void 18 in tibia 10 with plug 20. First, a surgeon accesses void 18. This step involves, for example, cutting into the patient's skin and cutting into soft tissue beneath the skin to reach tibia 10. Accessing void 18 may further involve removing an implant and its associated anchors from tibia 10 to expose void 18. Next, the surgeon screws plug 20 into void 18. Specifically, the surgeon engages bore 28 of proximal end 22 with a tool, such as a hex wrench, and screws distal end 24 into void 18. The surgeon may continue screwing plug 20 into void 18 until proximal end 22 of plug 20 sits substantially in line with or beneath cortical surface 16 of tibia 10.

According to an embodiment of the present invention, plug 20 may be a uni-cortical device. As a uni-cortical device, plug 20 is sized such that distal end 24 of plug 20 extends into cancellous layer 12 of tibia 10 when positioned within void 18. In other words, plug 20 enters cortical layer 14 of tibia 10 and extends into cancellous layer 12 of tibia 10 without reaching opposing cortical layer 14′ of tibia 10.

According to another embodiment of the present invention, plug 20 may be a bi-cortical device. As a bi-cortical device, plug 20 is sized such that distal end 24 extends through cancellous layer 12 and into opposing cortical layer 14′ of tibia 10 when positioned within void 18, as illustrated in FIG. 6. In other words, plug 20 enters cortical layer 14 of tibia 10 and extends through cancellous layer 12 of tibia 10 and into opposing cortical layer 14′ of tibia 10. The bone of cancellous layer 12 is soft and spongy compared to the bone of cortical layer 14, so driving a bi-cortical device into opposing cortical layer 14′ may provide a secure connection between plug 20 and tibia 10 and may reduce the risk of plug 20 loosening within void 18 over time.

According to yet another embodiment of the present invention, plug 20 may be a bi-cortical device designed to project beyond tibia 10. In this embodiment, plug 20 is sized such that distal end 24 of plug 20 extends through cancellous layer 12, into opposing cortical layer 14′, and beyond opposing cortical surface 16′ of tibia 10. In other words, plug 20 extends through tibia, from cortical surface 16 to opposing cortical surface 16′. As discussed above, the bone of cancellous layer 12 is soft and spongy compared to the bone of cortical layer 14, so driving a bi-cortical device into opposing cortical layer 14′ and beyond opposing cortical surface 16′ may provide a secure connection between plug 20 and tibia 10 and may reduce the risk of plug 20 loosening within void 18 over time.

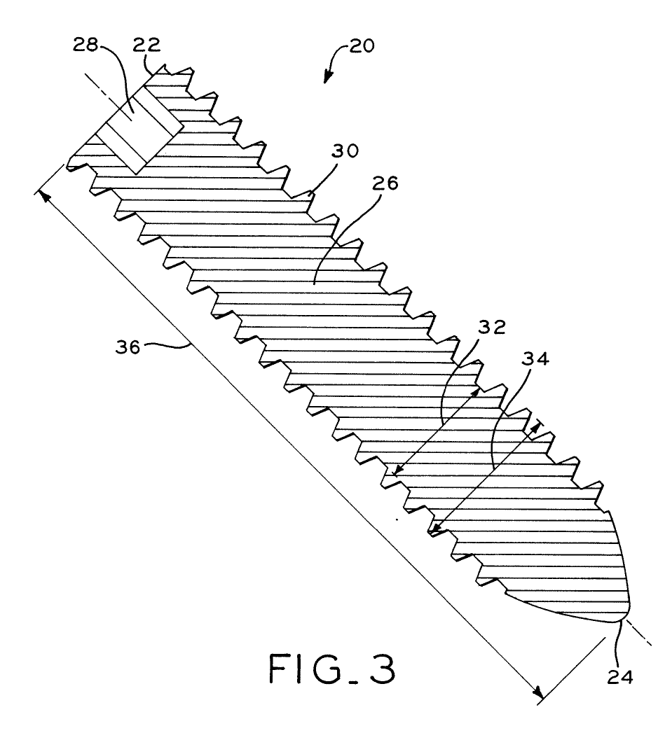

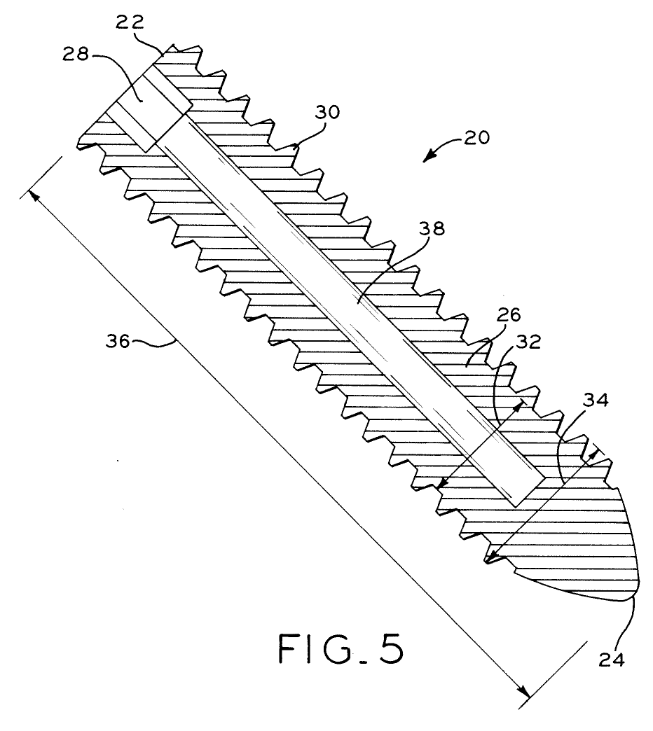

Plug 20 may be provided in various sizes to accommodate voids 18 of various sizes. For example, as shown in FIGS. 3 and 5, shaft 26 may have an outer diameter 32 of approximately 1-10 millimeters. Thread 30 may also be provided to extend various distances from shaft 26, such as approximately 1-5 millimeters. Therefore, plug 20, including shaft 26 and thread 30, may have an outer diameter 34 of approximately 2-15 millimeters, for example. Similarly, plug 20 may have a length 36 of several millimeters to several centimeters, and more specifically approximately 5-150 millimeters (0.5-15 centimeters). In an exemplary form of the present invention, the size of plug 20 slightly exceeds the size of the removed bone screw and void 18 to enhance fixation between plug 20 and the bone surrounding void 18. For example, outer diameter 32 of shaft 26 may essentially equal the diameter of void 18, while thread 30 may be sized to extend beyond shaft 26 and into the bone surrounding void 18. Similarly, length 36 of plug 20 may exceed the length of void 18, permitting distal end 24 of plug 20 to be driven into the bone surrounding void 18. As mentioned above, thread 30 is configured to tap the bone surrounding void 18 to provide a secure connection between plug 20 and tibia 10.

Referring again to FIGS. 2-5, plug 20 is constructed of a porous, open-cell material. As used herein, an “open-cell material” is a material containing pores that are connected to each other and form an interconnected network. Plug 20 may have a porosity as low as 55, 60, or 65 percent and as high as 80, 85, or 90 percent or more. In an exemplary embodiment of the present invention, plug 20 is constructed of a porous, open-cell metal to provide durability while also permitting bone growth into plug 20.

An example of such a material is produced using Trabecular Metal™ technology generally available from Zimmer, Inc., of Warsaw, Ind. Trabecular Metal™ is a trademark of Zimmer Technology, Inc. Such a material may be formed from a reticulated vitreous carbon foam substrate which is infiltrated and coated with a biocompatible metal, such as tantalum, by a chemical vapor deposition (“CVD”) process in the manner disclosed in detail in U.S. Pat. No. 5,282,861, the disclosure of which is expressly incorporated herein by reference. In addition to tantalum, other metals such as niobium, or alloys of tantalum and niobium with one another or with other metals may also be used.

Generally, the porous tantalum structure includes a large plurality of ligaments defining the open cells, or open spaces, therebetween, with each ligament generally including a carbon core covered by a thin film of metal such as tantalum, for example. The open spaces between the ligaments form a matrix of continuous channels having no dead ends, such that growth of cancellous bone through the porous tantalum structure is uninhibited. The porous tantalum may have a porosity as low as 55, 60, or 65 percent and as high as 80, 85, or 90 percent or more. Thus, porous tantalum is a lightweight, strong porous structure which is substantially uniform and consistent in composition, and closely resembles the structure of natural cancellous bone of cancellous layer 12, thereby providing a matrix into which cancellous bone may grow to provide fixation of plug 20 to tibia 10.

The porous tantalum structure may be made in a variety of densities to selectively tailor the structure for particular applications. In particular, as discussed in the above-incorporated U.S. Pat. No. 5,282,861, the porous tantalum may be fabricated to virtually any desired porosity and pore size, and can thus be matched with the surrounding natural bone to provide an improved matrix for bone ingrowth and mineralization.

Advantageously, plug 20 in this form is strong and durable enough to increase the strength of void 18 and the bone surrounding void 18 upon insertion into void 18. Also, the porous, open cell construction of plug 20 may increase the strength of void 18 and the bone surrounding void 18 by permitting bone growth into plug 20.

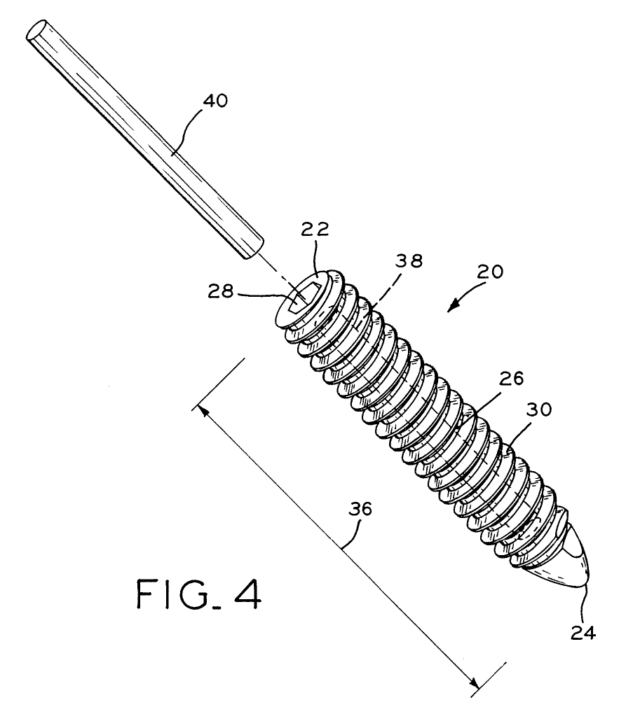

According to an embodiment of the present invention, illustrated in FIGS. 4-5, plug 20 is provided with a hollow interior, referred to herein as channel 38. Channel 38 may extend partially or entirely through plug 20. Channel 38 may extend entirely through plug 20 such that plug 20 is cannulated and capable of receiving, for example, a guide wire. Channel 38 may also be provided so that plug 20 is capable of receiving insert 40. Insert 40 may include a bone growth promoting material, medication, such as an antibiotic, or any other material capable of promoting healing and/or enhancing the strength of the bone surrounding void 18. A surgeon could select an appropriate insert 40 depending on the patient's particular needs. It is within the scope of the present invention that the size of channel 38 may vary as it extends through plug 20. For example, channel 38 may have a wide diameter near proximal end 22 to accommodate both insert 40 and a guide wire, and channel 38 may narrow near distal end 24 to prevent insert 40 from exiting plug 20 while still accommodating the guide wire.

An example of such a material is CopiOs™ Bone Void Filler generally available from Zimmer, Inc., of Warsaw, Ind. CopiOs™ is a trademark of Zimmer Spine, Inc. CopiOs™ Bone Void Filler contains calcium phosphate dibasic and osteoinductive bone morphogenetic proteins (BMPs). The material has a moderately acidic composition, which promotes the solubility of calcium and BMPs. The material also has a porous collagen scaffold to promote bone growth into insert 40. The presence of insert 40 may further enhance bone growth into porous plug 20.

While this invention has been described as having preferred designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A plug for filling a bone void comprising:
a distal end;
a proximal end;
a shaft extending between the proximal end and the distal end of the plug; and
a thread that extends from the shaft and wraps helically around the shaft, the plug comprising a porous, open-cell material.

2. The plug of claim 1, wherein the shaft is constructed entirely of the porous, open-cell material.

3. The plug of claim 1, wherein the open-cell material comprises tantalum.

4. The plug of claim 1, wherein the shaft has a porosity exceeding approximately 55 percent.

5. The plug of claim 1, wherein the thread is configured to tap bone surrounding the bone void.

6. The plug of claim 1, wherein the distal end of the plug is configured to tap cortical bone surrounding the bone void.

7. The plug of claim 1, wherein the thread extends from the distal end to the proximal end of the plug.

8. The plug of claim 7, wherein the proximal and distal ends define terminal ends of the plug, whereby the plug lacks a head that extends radially outwardly beyond the shaft.

9. The plug of claim 1, wherein the plug extends from the proximal end with an essentially uniform diameter until tapering near the distal end, whereby the proximal end of the plug lacks a head that extends radially outwardly beyond the shaft.

10. The plug of claim 1, wherein an outer diameter of the shaft is approximately 1-5 millimeters.

11. The plug of claim 1, wherein an outer diameter of the plug, including the shaft and the thread, is approximately 3-8 millimeters.

12. The plug of claim 1, wherein a length of the plug is approximately 8-150 millimeters.

13. The plug of claim 1, further comprising a channel within the shaft.

14. The plug of claim 13, further comprising a bone growth substance within the channel of the shaft.

15. The plug of claim 14, wherein the bone growth substance comprises calcium phosphate.

16. A plug for filling a void in a bone comprising:
a distal end defining a terminal end of the plug;
a proximal end defining another terminal end of the plug;
a shaft sized to be received within the void, the shaft extending between the distal end and the proximal end, whereby the plug lacks a head that extends radially outwardly beyond the shaft; and
a thread that extends from the shaft and wraps helically around the shaft, the plug comprising a porous, open-cell material.

17. The plug of claim 16, wherein the shaft is constructed entirely of the porous, open-cell material.

18. The plug of claim 16, wherein the shaft has a porosity exceeding approximately 55 percent.

19. The plug of claim 16, wherein the thread is configured to tap bone surrounding the void.

20. The plug of claim 16, further comprising:
a channel within the shaft; and
a bone growth substance positioned within the channel.

21. The plug of claim 16, wherein the shaft has a length sized to extend from a cortical layer of the bone to an opposing cortical layer of the bone.

22. A method of filling a void in a bone comprising the steps of:
providing access to the void; and
filling the void with a plug constructed of a porous, open-cell material and sized to be received within the void, said plug comprising a proximal end, a distal end, a shaft extending between the proximal end and the distal end, and a helical thread.

23. The method of claim 22, wherein the plug has a porosity exceeding approximately 55 percent.

24. The method of claim 22, wherein the step of filling the void comprises filling the void with the plug containing a bone growth substance within the plug.

25. The method of claim 22, wherein the step of filling the void comprises screwing the plug into the void until the proximal end of the plug is driven essentially in line with a cortical surface of the bone.

26. The method of claim 22, wherein the step of filling the void comprises screwing the plug into the void until the proximal end of the plug is driven into a cortical layer of the bone and the distal end of the plug is driven into an opposing cortical layer of the bone.

27. The method of claim 22, wherein the step of providing access to the void comprises removing an implant and exposing the void.
